(19) United States
(12) Patent Application Publication
(10) Pub. No.: US 2012/0136362 A1
(43) Pub. Date: May 31, 2012

(54) Framework for expanding bone

(76) Inventor: Kyung-Woo Park, Seoul (KR)

(21) Appl. No.: 13/136,248

(22) Filed: Jul. 27, 2011

(30) Foreign Application Priority Data
Nov. 26, 2010 (KR) 10-2010-0118657

(51) Int. Cl. A61B 17/58

(52) U.S. Cl. 606/92

(57) ABSTRACT

Provided is a bone expanding framework, which can secures complete reduction of a compressed bone tissue because elevation of the bone tissue is increased by a desired height without shrinking through a characteristic in which a spiral leaf spring having superelasticity is self-set in bone. The framework for expanding bone includes a spiral leaf spring which has a shape in which a thin and long band formed of a metal material is wound in a spiral shape, is inserted into the bone, and is expanded in the bone to reduce elevation of the compressed bone.

### BACKGROUND OF THE INVENTION

1. Field of the Invention

Exemplary embodiments of the present invention relate to a bone expanding framework for filling an inflatable body such as bone cement in a bone cavity to treat diseases such as fractures, osteoporosis, and non-osteoporosis of humans or animals, and more particularly, to a bone expanding framework, which can be easily inserted into retiform bone and self-set in bone.

2. Description of Related Art

Vertebral fractures in humans are associated with significant morbidity and mortality. Vertebral compression fractures (VCFs) frequently occur in patients suffering from a chronic disease such as osteopenia or osteoporosis that is a common disease of aged persons or an acute disease such as cancer. Particularly, there is a relatively high prevalence of osteoporotic VCFs in the elderly population, and especially in older women (i.e., aged 50 or older). VCFs are also common in patients on long-term steroid therapy, and in those suffering from multiple myeloma or cancers that have metastasized to the spine. Medical treatment of these fractures may include bed rest, orthotics, and analgesic medications. VCFs can also be caused by trauma, such as an automobile accident or falls. Medical treatment for traumatic fractures may include fusions and fixation with screws, rods and plates.

Vertebroplastry is a procedure in which bone cement is injected into a fractured vertebral body in an attempt to stabilize fractured segments and reduce pain. This procedure was originally used to treat spinal lesions caused by metastases and has recently been used to treat severe bone loss caused by osteoporosis (Eck et al. (March 2002) American J. Orthop. 31 (3): 123-127).

As shown in FIGS. 1A to 1C, typical VCFs includes anterior wedge fractures in which a cancellous bone tissue between discs is compressed in a front direction (see FIG. 1A), bi-concave fractures in which a central portion of a cancellous bone tissue is compressed (see FIG. 1B), and vertebra plana fractures in which the entire plane of a cancellous bone tissue is uniformly compressed (see FIG. 1C). When VCF is diagnosed, certain diagnostic or therapeutic procedures require the formation of a cavity in a bone mass. This procedure can be used to treat any bone, for example, bone which due to osteoporosis, avascular necrosis, cancer, or trauma, is fractured or is prone to compression fracture or collapse. These conditions, if not successfully treated, can result in deformities, chronic complications, and an overall adverse impact upon the quality of life.

For example, therapeutic procedures for the VCF are disclosed in U.S. Pat. Nos. 4,969,888 and 5,108,404 and Domestic Pat. Nos. 0889416 and 0920554. According to these patents, a therapeutic technology in which an expandable body is inserted into a cancellous bone tissue to artificially form an interior cavity in retiform bone, and then an inflatable body such as bone paste, cement, autograft material, allograft material, etc., is inserted into the cavity has been proposed as part of a therapeutic procedure for treating abnormal bone conditions such as fractures, osteoporosis, and non-osteoporosis of humans or animals.

FIGS. 2 and 3 are views illustrating a structure for installing an inflatable body that is called a balloon 102 in retiform bone described in Domestic Pat. Nos. 0889416 and 0920554.

The above-described structure includes a probe that introduces a passageway to a cancellous area, a cannula 100 which expands a hole in the bone and provides a passageway for a tamp to push or tamp a cancellous tissue, thereby forming a cavity (see FIG. 2), and a syringe which fills the cavity with an appropriate treatment material.

As shown in FIG. 3, according to Domestic Pat. Nos. 0889416 and 0920554, the cannula 100 is inserted into a cancellous bone tissue 112, and air is injected into the balloon 102 disposed at a tip end of the cannula 100 to inflate the balloon 102. Then, the cannula 100 is removed and cement 108 is injected through the tamp into the cavity 106 to tamp the cement into the cancellous bone tissue 112.

The inflatable body of the balloon structure used for treating VCFs through a series of processes has following limitations.

First, since the balloon expanded by the air pressure injected through the probe has a weak inflation force, it is difficult to construct a uniform balloon configuration. Thus, it is difficult to uniformly maintain density of the bone cement within the balloon. That is, since various variations on an inflation profile of upper/lower sides or lateral surfaces occur according to conditions of a patient, it is very difficult to allow a doctor to adequately adjust the density of the bone cement within the balloon according to the conditions of the patient. As a result, it is not easy to accomplish the precise procedure.

Second, even though the balloon is uniformed inflated, since the inflation of the balloon is different according to patients having bone tissues different from each other, for example, patients having hard bone and patients having soft bone.

Third, when the balloon inflation force is weak, there are limitations that a sufficient amount of cement is not injected, as well as, it is difficult to secure a required elevation (that represents a phenomenon in which the cancellous bone tissue shown in FIG. 1 returns to its original state from its crushed state).

Fourth, since a structure in which the cement is injected into the bone through the balloon structure is used for typical therapeutic procedures, a supporting force of the cement is very weak. That is, since the cement has a form similar to that of a nonreinforeced concrete structure, but a reinforced concrete structure in the construction industry, it is difficult to secure a sufficient cement strength. In addition, due to weak cement strength, there is a high possibility to require surgery again.

Fifth, when doctors use the inflatable bone tamp prepared by mixing polymer with the cement as a general inflatable structure, various tools are required to align the inflated surfaces and rotate the balloon along a precise track. Thus, the procedure may be complicated, and it may be difficult to locate the balloon at an optimal position.

### SUMMARY OF THE INVENTION

An embodiment of the present invention is directed to a bone expanding framework, which can secures complete reduction of a compressed bone tissue because elevation of the bone tissue is increased by a desired height without shrinking through a characteristic in which a spiral leaf spring having superelasticity is self-set in bone.

Another embodiment of the present invention is directed to a bone expanding framework, which can secure a sufficient supporting force because a leaf spring having a self-setting characteristic serves as a plate for reinforcing cement filled into bone.

Another embodiment of the present invention is directed to a bone expanding framework in which a spring leaf spring can be inserted into a portion to be undergoing surgery in a state where it is shrunken during procedure to a minimum diameter and be self-set in the inserted state to easily perform the procedure because the spiral leaf spring is manufactured using a nitinol alloy having a shape memory characteristic.

Another embodiment of the present invention is directed to a bone expanding framework in which a leaf spring can be precisely located at a desired position because the leaf spring is inserted into bone using a cannula.

Other objects and advantages of the present invention can be understood by the following description, and become apparent with reference to the embodiments of the present invention. Also, it is obvious to those skilled in the art to which the present invention pertains that the objects and advantages of the present invention can be realized by the means as claimed and combinations thereof.

In accordance with an embodiment of the present invention, a framework for expanding bone includes a spiral leaf spring which has a shape in which a thin and long band formed of a metal material is wound in a spiral shape, is inserted into the bone, and is expanded in the bone to reduce elevation of the compressed bone.

The spiral leaf spring may have a tip end with a streamline tip structure. Also, the framework may further include a shaft receiving space defined in a central portion of the spiral leaf spring, wherein a mechanism is inserted into the shaft receiving space to wind the spiral leaf spring.

Also, the spiral leaf spring may be wound to a minimum diameter at a temperature of about and be formed of a nitinol metal having a shape memory characteristic to return to its original shape at a temperature of a human body.

### BRIEF DESCRIPTION OF THE DRAWINGS

FIGS. 1A to 1C are views illustrating examples of typical vertebral compression fractures.

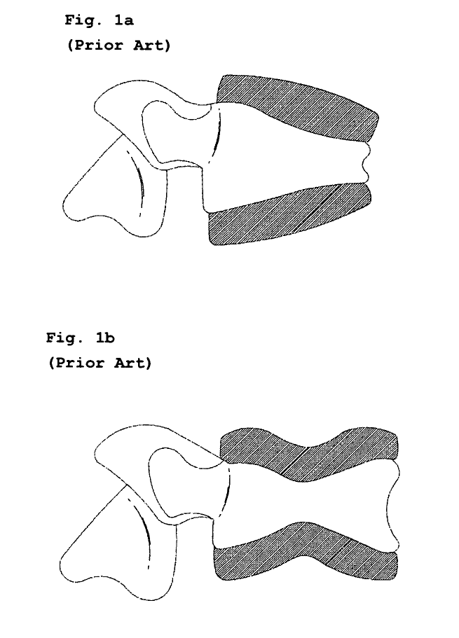

FIG. 2 is a view illustrating a structure of a cannula for inserting an inflatable body in bone in accordance with a related art.

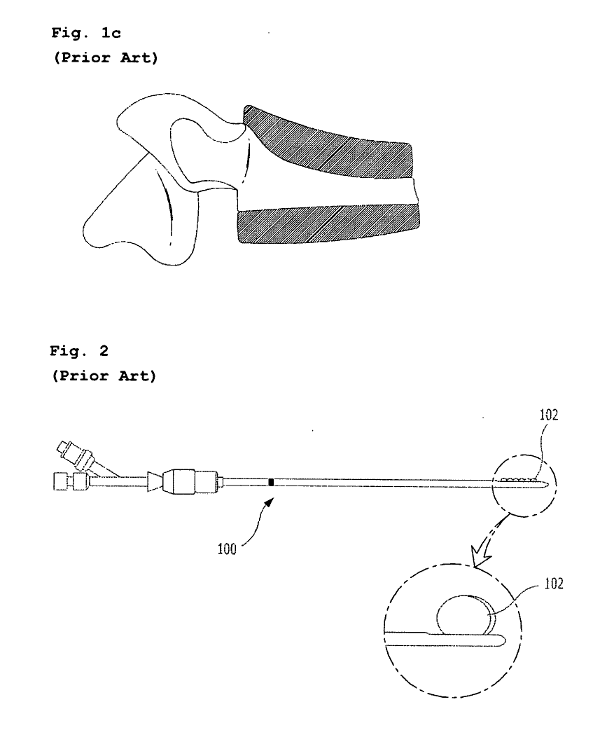

FIGS. 3A to 3C are views for explaining a process of filling bone cement using an inflatable body having a balloon structure as therapeutic procedures for vertebral compression fractures in accordance with the related art.

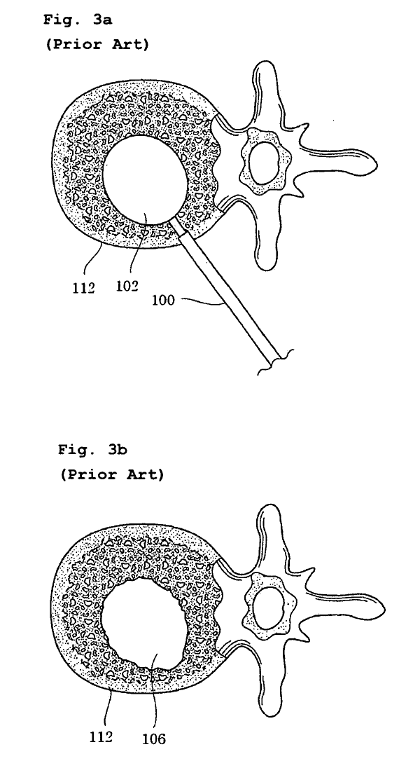

FIG. 4 is a perspective view of a framework for expanding bone in accordance with an embodiment of the present invention.

FIGS. 5A and 5b are front and side views of the framework for expanding the bone illustrated in FIG. 4.

FIG. 6 is a configuration view of a tool for inserting a spiral leaf spring into bone in accordance with the present invention.

### DESCRIPTION OF SPECIFIC EMBODIMENTS

Objects, other objects, characteristics and advantages of the present invention will be easily understood from an explanation of a preferred embodiment that will be described in detail below by reference to the attached drawings.

A framework for expanding bone in accordance with the present invention may be configured to increase an elevation to a desired height through a self-setting characteristic in bone without shrinking a bone tissue to secure complete reduction of the compressed bone tissue and a sufficient supporting force against a spine.

The bone expanding framework has a structure which is adequately applicable to vertebroplasty for treating vertebral compression fractures (VCFs) due to osteoporosis frequently occurring in the older generation.

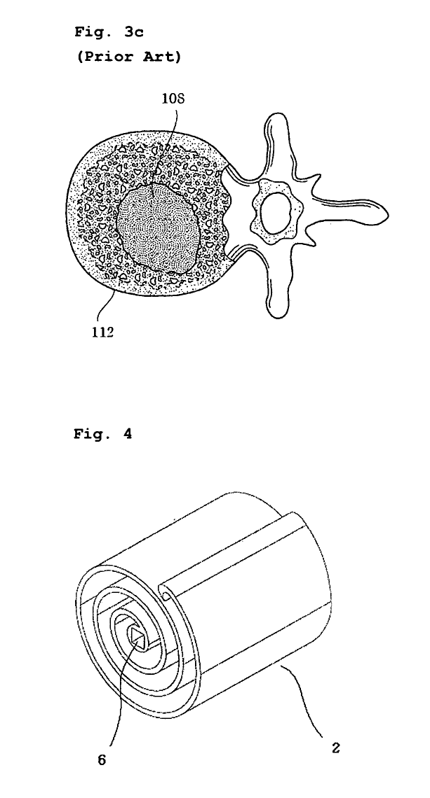

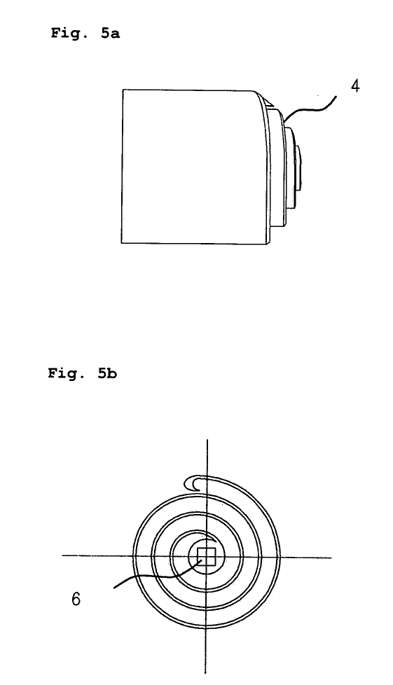

FIG. 4 is a perspective view of a framework for expanding bone in accordance with an embodiment of the present invention, and FIGS. 5A and 5b are front and side views of the framework for expanding the bone illustrated in FIG. 4.

As shown in FIG. 4, the bone expanding framework in accordance with the present invention includes a spiral leaf spring 2 having a shape in which a thin and long band formed of a metal material is wound in a spiral shape.

When the spiral leaf spring 2 is wound in a spiral shape substantially similar to a spring, the spiral leaf spring 2 is reduced in size. On the other hand, when the spiral leaf spring 2 is unwound, the spiral leaf spring 2 is expanded in size. The spiral leaf spring 2 is inserted into the bone. Then, the spiral leaf spring 2 is self-set in the bone to reduce the elevation of the compressed bone.

A tip end of the spiral leaf spring 2 includes a streamline tip 4 so that it is strongly and precisely inserted into cancellous bone. Also, a shaft receiving groove 6 in which a mechanism is fitted to wind the spiral leaf spring 2 is defined in a central portion of the spiral leaf spring 2.

The shaft receiving groove 6 may have a square, hexagonal, or octagonal shape to allow the mechanism to easily wind the spiral leaf spring 2.

In an embodiment of the present invention, the spiral leaf spring 2 is wound with a minimum diameter at a temperature of about 4° C. Also, the spiral leaf spring 2 is formed of a nitinol metal having a shape memory characteristic so that it is restored to its original shape at a temperature of a human body.

For example, in the above-described structure, when the spiral leaf spring 2 having a diameter of about 15 mm is immersed into cold water having a temperature of about 4° C., the spiral leaf spring 2 may be shrunk to a diameter of about 5 mm. Thus, the shrunk spiral leaf spring 2 may be fitted into a cannula having a diameter of about 6 mm.

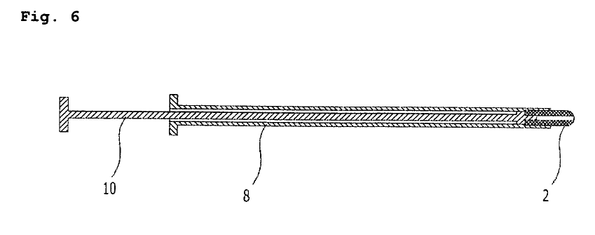

FIG. 6 is a configuration view of a tool for inserting a spiral leaf spring into bone in accordance with the present invention.

As shown in FIG. 6, the spiral leaf spring 2 is inserted into a front end of a holder 8. Also, a pressure bar 10 is inserted into the holder 8. A front end of the pressure bar 10 may have a square, hexagonal, or octagonal shape corresponding to that of the shaft receiving groove 6. In an embodiment of the present invention, the front end of the pressure bar 10 may include the cannula having a section, which is inserted into the shaft receiving groove 6 of the spiral leaf spring 2 to wind or unwind the spiral leaf spring 2.

An operation state for inserting the spiral leaf spring having the above-described constitutions according to the present invention into the bone will be described with reference to FIG. 6.

As shown in FIG. 6, the spiral leaf spring 2 is inserted into the front end of the holder 8, and the front end of the pressure bar 10 inserted into the holder 8 is inserted into the shaft receiving groove 6. Then, the pressure bar 10 is rotated to reduce a size of the spiral leaf spring 2. Here, when the spiral leaf spring 2 having a diameter of about 15 mm is immersed into cold water having a temperature of about 4° C., the spiral leaf spring 2 is shrunk by the shape memory characteristic thereof. As a result, the pressure bar 10 may be rotated to wind the spiral leaf spring 2, thereby easily shrinking the spiral leaf spring 2 to a desired size. Here, the spiral leaf spring 2 is maintained in the shrunk state without executing an elastic force thereof.

Also, the shrunk spiral leaf spring 2 is inserted into the holder 8 to enter into bone (cancellous bone). Then, the pressure bar 10 is separated from the holder 8. Since the spiral leaf spring 2 is inserted into the bone in a state where it is shrunk to a minimal size, an inflatable bone tamp may be easily located at a desired position.

The shrunk spiral leaf spring 2 may be restored in its original shape by a temperature of human body as time goes on. Here, the spiral leaf spring 2 is gradually expanded by the temperature of the human body to return to its original state before winding. Thus, since the spiral leaf spring 2 is self-set, the spiral leaf spring 2 forms a cavity while it uniformly applies a setting force into the bone. That is, the wound spiral leaf spring 2 is unwound by a restoring force thereof and expanded. Thus, the elevation of the caved bone may be increased by the self-setting force of the spiral leaf spring 2.

Although the spiral leaf spring 2 inserted into the bone is restored by the temperature of the human body, the spiral leaf spring 2 inserted into the bone may be repeatedly unwound and rewound using the pressure bar 10 (cannula) to increase the setting force of the spiral leaf spring 2.

Also, the spiral leaf spring 2 is located within the bone in the state where it is expanded. Then, the cannula is inserted into the holder 8 to inject bone cement. The bone cement is reinforced by the spiral leaf spring 2 to execute a strong supporting force to the spine.

In the operation state of the present invention, the elastic restoring force of the spiral leaf spring 2 is decided by various factors. That is, the elastic restoring force of the spiral leaf spring 2 is decided by factors such as a wound diameter of the spiral leaf spring 2, a spring size such as a thickness or width of a plate, the wound number, an unprocessed material shape, and the shape memory characteristic of a nitinol alloy. Thus, a good expanding force may be secured by synergy effects of a design and a material without shrinking. Specifically, the above-described factors deciding the elastic restoring force of the spiral leaf spring 2 may be adjusted to execute the self-setting force corresponding to patients having various bone tissues, for example, patients having hard bone tissues and patients having soft bone tissues, thereby adjusting a density of the bone cement.

The present invention having the above-described structure may be applicable to replacements annulus fibrosus of an intervertebral disc and nucleus pulposus of the intervertebral disc.

That is, after a resection of a disc is undergone, since the spiral leaf spring structure is compressed and inserted into porous annulus fibrosis and the disc is increased in height by the restoring force of the spiral leaf spring 2, back pains or sciatrica due to a recurrence of disc herniation (prolapse) may be avoided, and also,

The recurrence after the disc surgery may be prevented.

Also, in a typical disc surgery, the inflatable disc tamp (nucleus pulposus) is inserted into a center of the intervertebral disc to reconstruct an intradical elasticity or an intradical pressure. However, according to the present invention, since the spiral leaf spring instead of the nucleus pulposus is used, the back pains or sciatrica may be more effectively treated.

As described above, the effects of the present invention may be classified into functional perspective and procedural perspective.

The effects in the functional perspective are as follows.

First, since the spiral leaf spring has the structure wound in the spring shape, the spiral leaf spring having the minimal size can be inserted into the bone. Also, since the spiral leaf spring is self-set to a strong expanding force, the expanding pressure within the bone can be uniformly distributed. That is, spiral leaf spring can be expanded at a uniform pressure in bones of different patients which have hard bone and soft bone, respectively. Thus, the elevation can be secured by a distance in which the spine of the patient is caved. Therefore, the compressed bone tissue can be completely reduced.

Second, since the spiral leaf spring together with the cement filled into the bone serves as reinforcements, the treated vertebrae can have a firm strength similar to that of the reinforced concrete structure, as well as, the sufficient supporting force can be applied to the spine. The above-described functions together with the spiral leaf spring can execute the strong supporting force in the bone against a compressing force in various axis directions after the bone cement is cured. Thus, the spiral leaf spring together with the cement can secure the firm supporting force in the VCFs, osteoporosis, and non-osteoporosis to easily reconstruct the spine.

Third, the spiral leaf spring can have the good expanding force, as well as, have various sizes such as a diameter, a length, and a thickness and various sections such as a circle and an oval. Thus, the inflatable bone tamp having flexibly curved shape can be applied according to the conditions of patients to uniformly adjust the density of the bone cement.

The effects in the procedural perspective are as follows.

First, the present invention can be applied to various bones including the spine, the radial, the humerus, the thighbone, the tibia, and calcaneus. However, the present invention is not limited to the foregoing bones.

Second, since the spiral leaf spring formed of the nitinol alloy having the shape memory characteristic is wound in the spring shape, the spiral leaf spring may have minimum diameter and height at a temperature of about 4° C. during the procedure. Also, since the spiral leaf spring shrunk in the minimum size can be mounted on cannula and inserted into the bone, the spiral leaf spring can be easily located at a position expandable in the bone. In addition, since the bone is punched with a minimal diameter, the procedure for inserting the spiral leaf spring from a rear side of the spine into the bone can be easily performed.

Third, since the tip end of the spiral leaf spring has the streamline tip structure, the spiral leaf spring can be strongly and precisely inserted into the cancellous bone.

Fourth, since the spiral leaf spring is formed of the nitinol alloy having the shape memory characteristic and freely rewound or unwound according to the conditions of patients, the inflatable bone tamp may be easily located at a desired position. In addition, when the spiral leaf spring is inserted, worry about a malposition of the spiral leaf spring can be dispelled.

While the present invention has been described with respect to the specific embodiments, it will be apparent to those skilled in the art that various changes and modifications may be made without departing from the spirit and scope of the invention as defined in the following claims.

1. A framework for expanding bone, comprising a spiral leaf spring which has a shape in which a thin and long band formed of a metal material is wound in a spiral shape, is inserted into the bone, and is expanded in the bone to reduce elevation of the compressed bone.

2. The framework of claim 1, further comprising a shaft receiving space defined in a central portion of the spiral leaf spring,
wherein a mechanism is inserted into the shaft receiving space to wind the spiral leaf spring.

3. The framework of claim 1, wherein the spiral leaf spring has a tip end with a streamline tip structure.

4. The framework of claim 1, wherein the spiral leaf spring has a shape memory characteristic.

5. The framework of claim 1, wherein the spiral leaf spring is wound to a minimum diameter at a temperature of about 4° C. and is formed of a nitinol metal having a shape memory characteristic to return to its original shape at a temperature of a human body.
